(19) United States
(12) Patent Application Publication
(10) Pub. No.: US 2013/0046220 A1
(43) Pub. Date: Feb. 21, 2013

(54) Wearable Wrist Support for Computer Mouse Users

(71) Applicant: Alanna Berthelot, Plaquemine, LA (US)

(72) Inventor: Alanna Berthelot, Plaquemine, LA (US)

(21) Appl. No.: 13/213,276

(22) Filed: Aug. 19, 2011

(51) Int. Cl. A61F 5/00

(52) U.S. Cl. 602/21

(57) ABSTRACT

A wearable wrist support comprising: an elongated strap, where the elongated strap wraps around a wrist of a user; a pocket positioned within a central portion of the elongated strap, where the pocket includes an opening at an end; a support member inserted within the pocket, where the support member comforts and elevates the wrist of the user; and a fastening means attached to an end of the elongated strap, wherein the fastening means secures the elongated strap around the wrist. The support member may be filled with a material that cushions the user's wrist. Alternatively, the support member may be filled with a material that may be heated or chilled to provide a therapeutic quality to the wearable wrist support.

### BACKGROUND OF THE INVENTION

1. Field of the Invention

The present invention relates to a wrist support for a user to wear around their wrist to cushion the wrist and hand while they are utilizing a mouse for their computer.

2. Description of Related Art

Nowadays, many people spend a majority of their day typing and interacting on a computer. Due to the increased amount of time with their wrist extended at unnatural and uncomfortable positions, many of these computer users have developed hand, wrist and arm injuries that often affect their work product. At times, the injuries become so bad that they require surgery to alleviate the pain caused by poor wrist placement. As a remedy to the wrist injuries, wrist guards and wrist pads have been developed which allow the computer users to rest, cushion and elevate their hands while using a keyboard. Most common are the wrist pads that stretch across the bottom of a keyboard that enable a user to position their wrist while they are typing.

Recently, wrist cushions that protect a user's wrist while moving a mouse have been developed. For example, U.S. Pat. No. 5,562,270 by Dale Montague discloses a wrist pad wherein a padded wrist rest is added to a standard mouse pad. The rest provides a cushion for the user while manipulating their mouse. U.S. Pat. No. 6,585,198 by Carmen Dillon discloses a wrist support for use with a computer mouse. The wrist support provides an ergonomic pad that follows the shape of a users wrist, palm, pinky and thumb for increased comfort while the user moves their mouse. Further, U.S. Pat. No. 7,183,523 by Chiang-Tsun Lu discloses a wrist rest equipped with a heating device. The wrist rest includes a heating element within the base that may be activated with a switch on the side of the rest. Alternatively, U.S. Pat. No. 6,082,682 by Vincent So et al. discloses a mouse user wrist support where the wrist support attaches around the user's wrist. The wrist support secures a cushion below the user's wrist for protection while using a mouse. While these wrist protectors do provide support for the user, none of them directly address comforting users who may already have injuries due to excessive computer use.

It would be beneficial in the art to provide a wrist support that may be worn by the user. It would desirable in the art to provide a wrist support that not only cushions but also provides additional relief for those persons already suffering with wrist ailments.

### SUMMARY OF THE INVENTION

In view of the foregoing disadvantages inherent in the prior art, the general purpose of the present invention is to provide a wearable wrist support, configured to include all of the advantages of the prior art, and to overcome the drawbacks inherent therein.

Accordingly, an object of the present invention is to provide a wrist support that allows a user to wear the support while they manipulate a computer mouse.

Another object of the present invention is to provide a wearable wrist support where the wrist supports are removable.

Further, another object of the present invention is to provide a wearable wrist support wherein the insertable wrist supports may be heated or cooled for additional therapeutic relief to those computer users with hand, wrist and arm injuries.

To achieve the above objects, in an aspect of the present invention, a wearable wrist support is described comprising: an elongated strap, where the elongated strap wraps around a wrist of a user; a pocket positioned within a central portion of the elongated strap, where the pocket includes an opening at an end; a support member inserted within the pocket, where the support member comforts and elevates the wrist of the user; and a fastening means attached to an end of the elongated strap, wherein the fastening means secures the elongated strap around the wrist. The support member may be filled with a material that cushions the user's wrist. Alternatively, the support member may be filled with a material that may be heated or chilled to provide a therapeutic quality to the wearable wrist support.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawings, wherein like elements are identified with like symbols, and in which:

FIG. 1 depicts a perspective view of a wearable wrist support in accordance with an exemplary embodiment of the present invention; and

FIG. 2 depicts a perspective view of a wearable wrist support worn on a user's wrist in accordance with an exemplary embodiment of the present invention.

Like reference numerals refer to like parts throughout the description of several views of the drawings.

### DETAILED DESCRIPTION OF THE DRAWINGS

The present invention relates to a padded bracelet wherein a wearable wrist support provides cushion and comfort to users while manipulating a computer mouse. The wearable wrist support is made from a breathable fabric that wraps securely around the user's wrist. The wearable wrist support includes a pocket to receive a removable wrist support, where the wrist supports are filled with variety of materials. For example, the wrist support may be filled with foam for a soft comfortable feel during use. Alternatively, the wrist supports may be filled with a material able to withstand heating and chilling for a therapeutic purpose. Therefore the wearable wrist support affords the users with a protective brace that may aid or prevent injuries incurred during extended wrist movement.

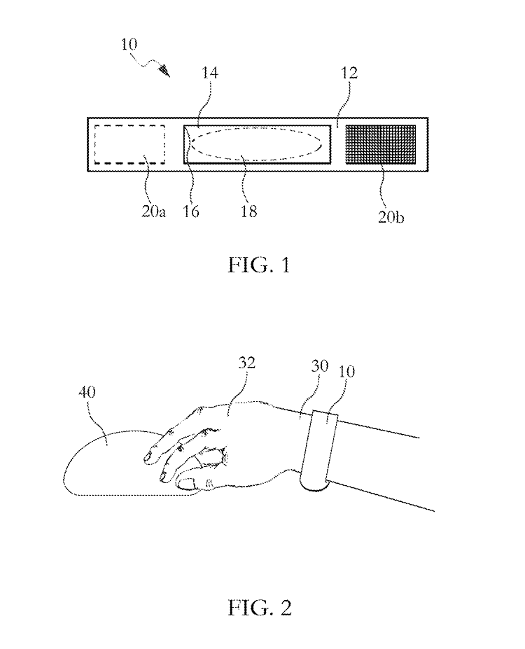

Turning now descriptively to the drawings, referring to FIG. 1, a perspective view of a wearable wrist support (10) is shown in accordance with an exemplary embodiment of the present invention. The wearable wrist support (10) includes an elongated strap (12) that fits comfortably around a user's wrist. The elongated strap (12) may be made from a soft, breathable and resilient material like cotton cloth, jean, canvass, nylon, polyester, suede, leather or another common material known and used in the art. The elongated strap (12) may be a single, double or triple layer fabric with reinforced edges to ensure durability and device longevity.

In a central portion of the elongated strap (12) may be a pocket (14). The pocket (14) may include an opening (16) to receive a support member (18). The opening (18) may be along a side (as shown) or along the top or bottom of the pocket (14). The opening (18) enables the user to easily insert and remove the support member (18). The material comprising the pocket (14) may be made from the same material as the elongated strap (12), and the pocket (14) may be attached to the elongated strap (12) with stitches, glue, hook and loop fasteners (commonly known as Velcro®) or the like.

The support member (18) may be a shaped insert that comfortably rests within the pocket (14) and against the user's wrist. The support member (18) may be shaped as an elongated oval (as shown), cylinder, rectangle or chain of circular bubbles attached together and easily inserted into the pocket (14). The support member (18) may be covered in a plastic or fabric material, and may be thereby filled with a variety of different materials including foam, small beads, cotton or synthetic stuffing, gel, saline, silicone, or alternative materials known and used able to withstand hot and freezing temperatures. Multiple support members (18) made from differing materials may be provided with each elongated strap (12) to enable the user to interchange the type of support member (18) desired for their particular needs.

During use the support member (18) may be used to simply cushion and elevate the user's wrist when manipulating a mouse. Alternatively, the support member (18) or the whole wearable wrist support (10) may be placed in a refrigerator or freezer to chill or freeze a cold withstanding material within the support member (18), thereby providing a chilled support member. Further yet, the support member (18) or the wearable wrist support (10) may be placed in a microwave to heat a heat withstanding material within the support member (18), thereby providing a heated support member. With the enhanced features of being able to not only support and cushion the wrist but also deliver the therapeutic effects of heat or cold to the wrist, the wearable wrist support (10) is especially beneficial for those users who suffer from ailments like carpal tunnel syndrome or strained muscles.

At the ends of the elongated strap (12) is a fastening means (20a, 20b). The fastening means (20a, 20b) may be a hook and loop fastener (as shown), clip, button, snap or the like. The fastening means (20a, 20b) enables the user to fasten the wearable wrist support (10) with a single hand around their mouse using wrist to easily attach and detach the elongated strap (12) at will.

Referring now to FIG. 2, a perspective view of the wearable wrist support (10) on a user's wrist (30) in accordance with an exemplary embodiment of the present invention. The wearable wrist support (10) may rest against a portion of the user's wrist (30) where the wrist (30) meets the hand (32). This way the user is able to maneuver the mouse (40) without constraint or restriction. And, since the wearable wrist support (10) is continuously attached to the user's wrist (30), the user never has to worry about repositioning the support. The wearable wrist support (10) may be utilized by the user from one mouse (40) to another, making it easily transportable, storable and comforting. In addition to using the wearable wrist support (10) while computing, the wearable wrist support (10) may be used while using the keyboard, writing, drawing, painting, drafting or any other activities where the user rests their wrists (30) or arms while their hand (32) is active. A pair of wearable wrist supports (10) may be utilized while typing on a keyboard to provide support and elevation to both of the user's hands (32) and wrist (30), instead of just the single mouse using hand.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A wearable wrist support comprising:
an elongated strap, where said elongated strap wraps around a wrist of a user;
a pocket positioned within a central portion of said elongated strap, where said pocket includes an opening at an end;
a support member inserted within said pocket, where said support member comforts and elevates the wrist of the user; and
a fastening means attached to an end of said elongated strap, wherein said fastening means secures said elongated strap around the wrist.

2. The wearable wrist support according to claim 1, wherein said support member is filled with a material.

3. The wearable wrist support according to claim 2, wherein said material is one of at least foam, beads, stuffing, gel, saline and silicone

4. The wearable wrist support according to claim 2, wherein said material is microwavable to provide a heated support member.

5. The wearable wrist support according to claim 2, wherein said material is freezable to provide a chilled support member.

6. The wearable wrist support according to claim 1, wherein the fastening means is one of at least a hook and loop fastener, a clip, a button and a snap.

7. The wearable wrist support according to claim 1, wherein said elongated strap is made from a material made from one of at least cotton cloth, jean, canvass, nylon, polyester, suede and leather.

8. The wearable wrist support according to claim 1, wherein said support member is shaped as one of at least an oval, a cylinder, a rectangle and a chain of circular bubbles.

9. The wearable wrist support according to claim 1, wherein said wearable wrist support is available as a pair to wrap around each wrist of the user.
